(19) United States
(12) Patent Application Publication
(10) Pub. No.: US 2009/0238341 A1
(43) Pub. Date: Sep. 24, 2009

(54) IMAGING SUPPORT DEVICE FOR RADIOGRAPHIC LONG LENGTH IMAGING

(75) Inventors: Takahiro KAWAMURA, Ashigarakami-gun (JP); Yoshitaka YAMAGUCHI, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/407,540

(22) Filed: Mar. 19, 2009

(30) Foreign Application Priority Data
Mar. 21, 2008 (JP) 2008-073746

(51) Int. Cl. H05G 1/28; A61B 6/08

(52) U.S. Cl. 378/162; 378/205

(57) ABSTRACT

An image taking support device for radiographic long length imaging, the image taking support device providing an image of a subject, the image being longer than a length of a maximum field of view of a radiation detector, by combining a plurality of radiographic images each partially overlapping another at the overlapping parts, the device comprising:
a supporting member including a radiotransparent material and supporting the subject;
markers including a substance with a large radiation attenuation coefficient, the markers being movably arranged along a surface of the supporting member supporting the subject so that the markers provide marks for combining the images;
an irradiated area determination device that determines an area to be irradiated with a radiation;
a marker position determining device that determines positions for arranging the markers, based on the determined area to be irradiated; and
a marker moving device that moves the markers to the determined positions of the markers.

### BACKGROUND OF THE INVENTION

1. Field of the Invention

The present invention relates to an image taking support device for radiographic long length imaging, and specifically relates to an image taking support device for radiographic long length imaging using an FPD (flat panel detector), the device being used for, when connecting a plurality of images obtained by performing image-taking while changing the radiation field of view, correctly aligning the connection positions of the images.

2. Description of the Related Art

Conventionally, radiographic apparatuses that apply radiation, such as an X-ray, to a subject and detect radiation passing through the subject to obtain a radiographic image for diagnosis have widely been known. Among these radiographic apparatuses, for example, one using a radiographic film, or a storage phosphor sheet that stores radiation energy, as a radiation detector that detects radiation passing through the subject has been known.

Also, in radiographic image taking for medical diagnosis, a long length of image is taken for figuring out and examining the entire spine or the entire lengths of the lower extremities of a subject at one time.

Conventionally, a long length of image is taken by setting a long length of radiographic film in a long length of dedicated cassette or arranging a plurality of storage phosphor sheets so as to partially overlap one another and setting them in a long length of dedicated cassette.

Also, in recent years, a flat panel-type X-ray detector (FPD: flat panel detector) using, e.g., semiconductor, in which numerous X-ray detection elements are arranged in a matrix on an X-ray detection surface has been known as an X-ray detector that detects an X-ray transmission image of a subject generated as a result of X-ray application via a X-ray tube.

In such long length imaging using a FPD, a long length of image is obtained by making the X-ray tube and the X-ray detector move in coordination with each other, obtaining plural X-ray images while changing the X-ray's field of view, and connecting these plural X-ray images.

In this case, markers that provide marks for alignment are shown in the overlapping parts of the images together with the subject. Then, when connecting the plural images, alignment is performed based on these markers and the image frames are automatically combined. The markers are formed of a material with a large X-ray attenuation, such as lead, for example, and arranged at positions where they do not overlap the image of the subject between an X-ray source and the X-ray detector (FPD).

Where performing long length imaging involving taking plural images as described above, the posture of a subject (patient) may change during the X-ray detector and the X-ray tube moving between image takings, causing inconvenience for image combination.

Therefore, conventionally, in order to avoid this inconvenience, art in which a screen is provided in front of the X-ray detector and a patient leans against the screen, thereby suppressing changes of the posture of the patient is known (see, for example, Japanese Patent Application Laid-Open No. 2005-278812).

### SUMMARY OF THE INVENTION

However, although in the above conventional art, the accuracy in obtaining a long length of image by combining plural images is enhanced by performing alignment using markers, in long length imaging using an FPD, the relationship between the markers and the FPD is not fixed and the X-ray's field of view is changed for each patient subject to image taking, and thus, it is necessary that the positions to arrange the markers be set within the overlapping areas of the images to be connected, the areas not overlapping the subject (patient), and furthermore, be set at positions in which the markers do not run out of the image, resulting in a problem in causing a lot of trouble for long length imaging.

The present invention has been made in view of the aforementioned circumstances, and an object of the present invention is to provide an image taking support device for radiographic long length imaging enabling easy arrangement of markers that provide marks for combining plural images when taking a long length of image using a FPD, saving trouble in long length imaging.

In order to achieve the aforementioned object, a first aspect of the present invention provides an image taking support device used for radiographic long length imaging, the image taking support device providing an image of a subject, the image being longer than a length of a maximum field of view of a radiation detector, by combining a plurality of radiographic images each partially overlapping another at the overlapping parts, the device comprising: a supporting member including a radiotransparent material and supporting the subject; markers including a substance with a large radiation attenuation coefficient, the markers being movably arranged along a surface of the supporting member supporting the subject so that the markers provide marks for combining the images; an irradiated area determination device that determines an area to be irradiated with a radiation; a marker position determining device that determines positions for arranging the markers, based on the determined area to be irradiated; and a marker moving device that moves the markers to the determined positions of the markers.

Consequently, in long length imaging using an FPD, markers can reliably be included within the irradiated area regardless of the image taking target, the arrangement of the markers, which provide marks for combining plural images, can easily be performed, and thus, the rate of failure of alignment for image combination to obtain a long length of image can be reduced, enabling provision of a proper automatic image combination result.

Also, since an image-taking person does not need to set the positions of the markers, the time for image taking can be reduced, and thus, the work efficiency can be enhanced, enabling saving trouble in long length imaging, which has conventionally been cumbersome.

Also, a second aspect of the present invention provides the image taking support device for radiographic long length imaging according to the first aspect, wherein the subject is arranged in a standing position.

Consequently, a long-length of image can easily be taken even when the subject is standing.

A third aspect of the present invention provides the image taking support device for radiographic long length imaging according to the second aspect, wherein: the markers are arranged substantially parallel to a body axis of the subject; a predetermined number of the markers is arranged on each of two rod-like members that are moved in parallel to each other to widen/narrow the space between the two rod-like members, with a predetermined space between each marker; and the marker moving device is a device that moves the two rod-like members on which the markers are arranged, in parallel to each other.

Consequently, the arrangement of the markers can easily be performed.

A fourth aspect of the present invention provides the image taking support device for radiographic long length imaging according to any of the first to third aspects, wherein the irradiated area determining device determines the area to be irradiated by calculating the area to be irradiated from a distance between a source of the radiation and the radiation detector and the size of an aperture for the radiation.

Consequently, a field of view can be figured out by calculation, and thus, the positions of the markers can automatically be moved to proper positions according to the width of the field of view.

A fifth aspect of the present invention provides the image taking support device for radiographic long length imaging according to the third aspect, further comprising: an irradiation field lamp arranged at a position that is substantially the same as the position of a source of the radiation, the irradiation field lamp applying visible light to the subject; and a plurality of photodetectors arranged on the rod-like members, wherein the irradiated area determining device detects light from the radiation field lamp while moving the rod-like members in parallel to each other, to determine the area to be irradiated.

Consequently, all the procedures from setting of the positions of the markers to movement of those positions can automatically performed, enabling further enhancement of work efficiency in long length imaging.

As described above, according to the present invention, in long length imaging using an FPD, markers can reliably be included within the irradiated area regardless of the image taking target, the arrangement of the markers, which provide marks for combining plural images, can easily be performed, and thus, the rate of failure of alignment for image combination to obtain a long length of image can be reduced, enabling provision of a proper automatic image combination result.

Also, since an image-taking person does not need to set the positions of the markers, the time for image taking can be reduced, and thus, the work efficiency can be enhanced, enabling saving trouble in long length imaging, which has conventionally been cumbersome.

### BRIEF DESCRIPTION OF THE DRAWINGS

FIG. 1 is a schematic diagram of a configuration of a first embodiment of a radiographic apparatus using an image taking support device for radiographic long length imaging according to the present invention;

FIGS. 2A and 2B are side views illustrating taking a long length of image of a spine using a radiographic apparatus according to the first embodiment;

FIG. 3 is a front view schematically illustrating a screen and a marker device;

FIGS. 4A to 4E are illustrations of example marker shapes;

FIG. 5 is a perspective view illustrating a specific example of a marker moving device;

FIG. 6 is a schematic diagram of a configuration of a second embodiment of a radiographic apparatus using an image taking support device for radiographic long length imaging according to the present invention; and

FIGS. 7A and 7B are illustrations of movement of the positions of markers.

### DETAILED DESCRIPTION OF THE PREFERRED EMBODIMENTS

Hereinafter, embodiments of an image taking support device for radiographic long length imaging according to the present invention will be described in details with reference to the accompanying drawings.

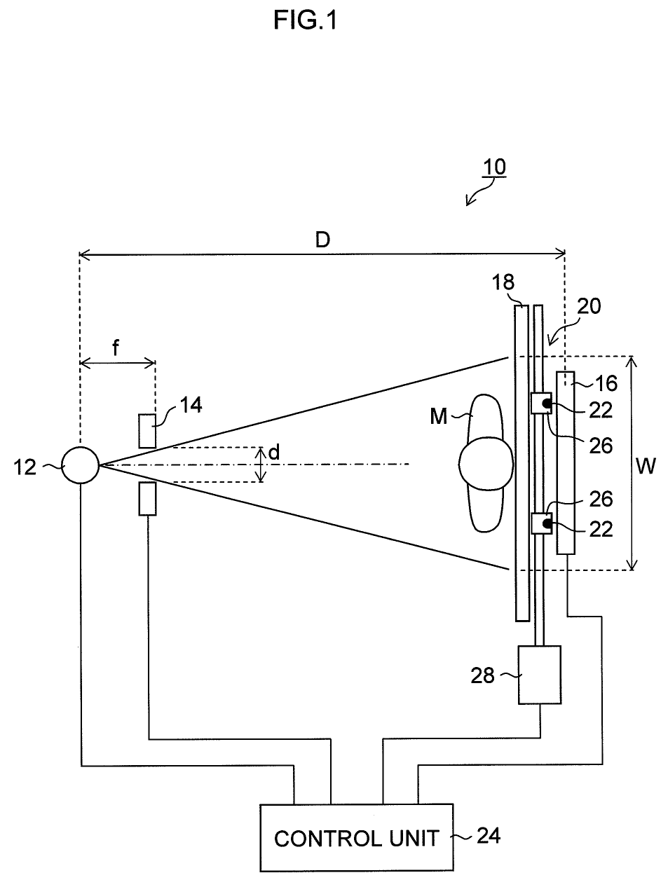

FIG. 1 is a schematic diagram of a configuration of a first embodiment of a radiographic apparatus using an image taking support device for radiographic long length imaging according to the present invention.

As illustrated in FIG. 1, a radiographic apparatus 10 according to the present embodiment mainly includes: an X-ray source 12 that applies an X-ray to a subject M; a collimator 14 that focuses an X-ray emitted from the X-ray source 12; a flat panel-type X-ray detector (FPD: flat panel detector) 16 that detects an X-ray that has passed through the subject M and outputs detection signals; a marker device 20 including a screen 18 and markers 22 that provide marks for combining plural images, which is included in a taking support device for long length imaging; and a control unit 24 that controls the aforementioned components.

Although a description of the detailed configuration will not be provided, the X-ray source 12 includes an X-ray tube that applies an X-ray to the subject M, and a tube voltage and a tube current provided to the X-ray tube are controlled by the control unit 24, thereby X-ray energy emitted being controlled.

The FPD 16 has a light-receiving surface of a surface thereof, the light-receiving surface being formed to be a flat plane, and the orientation of the light-receiving surface can be changed so as to be horizontal or vertical. The FPD 16 is used in such a manner that it is provided behind the back of the subject M, and photoelectrically detects an X-ray that has passed through the subject M, and outputs analog electrical signals. The output signals from the FPD 16 are input to the control unit 24 and converted into digital signals in the control unit 24, and subjected to image processing. Although not illustrated, the FPD 16 is constructed in such a manner that it can move along a supporting column provided in parallel to the body axis of the subject M.

The screen 18 is provided for holding a posture of the subject M during taking a plurality of images of the subject M in a standing position, like when taking a long length of image of, e.g., the entire spine, for example. The screen 18 is formed of a radiotransparent material.

The marker device 20 is provided for setting the markers 22, which provide marks for combining plural images, to proper positions depending on the subject M and the radiographic image.

The markers 22 are provided on marker holding members 26 and can be moved by marker moving devices 28 in a direction of the width of an X-ray's field of view indicated by reference character W.

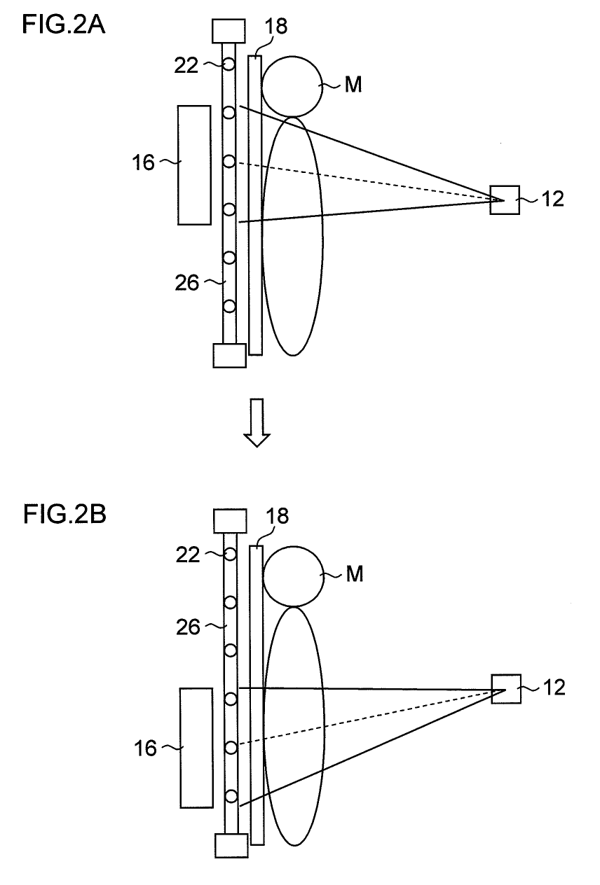

FIGS. 2A and 2B are side views illustrating taking a long length of image of a spine using the radiographic apparatus 10.

First, as illustrated in FIG. 2A, when the subject M stands with his/her back against the screen 18, the FPD 16 is set in a position enabling detection of an X-ray that has passed through the chest of the subject M, by means of a moving device (not illustrated). Next, the area to be irradiated with an X-ray (field of view) is calculated, and the marker holding members 26 are moved to the edge portions in the lateral direction of the area to be irradiated, by means of the marker moving devices 28. Then, an X-ray is applied toward the chest of the subject M from the X-ray source 12 to take an image of the upper half of the spine.

Detection signals that have entered the FPD 16 and detected by the FPD 16 are sent to the control unit 24. The control unit 24 converts the received detection signals to digital signals to generate image data and temporarily stores it in memory (not illustrated).

Next, as illustrated in FIG. 2B, the FPD 16 is moved downward to set in a position enabling detection of an X-ray that has passed through the abdomen of the subject M. Then, the direction of the X-ray applied from the X-ray source 12 is changed with the positions of the markers 22 unchanged to apply the X-ray toward the abdomen of the subject M, thereby taking an image of the lower half of the spine.

The signals detected by the FPD 16 are sent to the control unit 24 and converted into digital signals to generate image data. This image data is also temporarily stored in the memory. Each of these radiographic images includes images of the markers 22 at its part overlapping the other radiographic image, together with an image of the subject M.

Then, in the control unit 24, the image of the upper half of the spine that has been taken first and the image of the lower half of the spine that has been taken later are read from the memory, and aligned based on the markers 22 shown within the overlapping parts of the images to combine the two images, thereby providing one long-length image showing the entire spine.

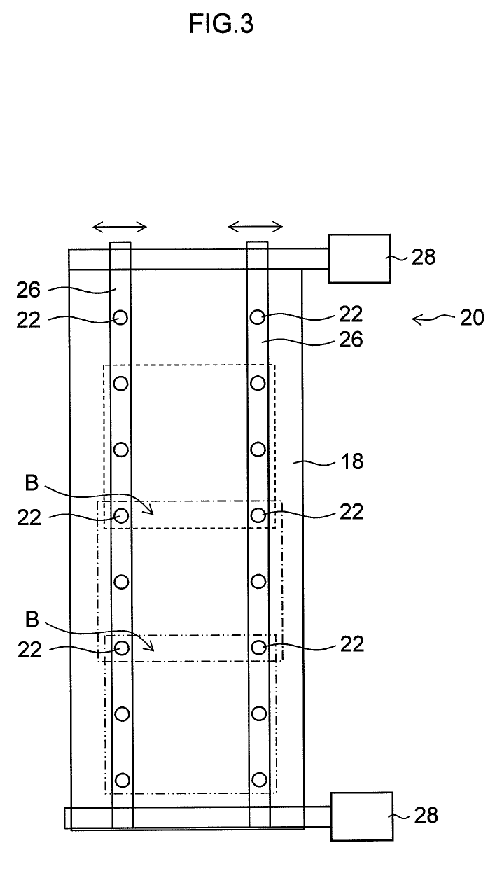

FIG. 3 is a schematic front view of the screen 18 and the marker device 20.

As illustrated in FIG. 3, the screen 18 is set up on a base (not illustrated), and is formed in the shape of a flat plate. It should be understood that the screen 18 is formed of a radiotransparent material.

Also, the back side of the screen 18 is provided with the marker holding members 26 parallel to each other, the marker holding members 26 being rod-like members bridging the marker moving devices 28 provided at the upper and lower portions. On the marker holding members 26, a plurality of the markers 22 are arranged at regular intervals. As indicated with arrows in the Figure, the two marker holding members 26 are formed so that they can be moved laterally in parallel to each other by means of the marker moving devices 28. An illustration of, e.g., members for supporting the marker moving devices 28 is omitted.

Also, in the Figure, three areas irradiated with an X-ray (fields of view) are illustrated with dotted lines, alternate long and short dashed lines and chain double-dashed lines. As described above, irradiated areas are set so that the markers 22 are included in the overlapping parts B of two irradiated areas B.

Although not illustrated in FIG. 3, images are taken by applying an X-ray with the FPD 16 arranged behind the position corresponding to each irradiated area. In the example illustrated in FIG. 3, three images are taken.

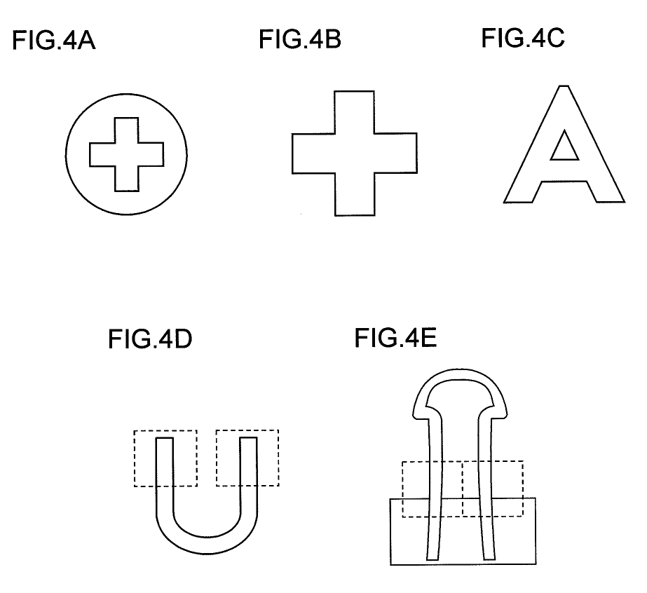

FIGS. 4A to 4E illustrate example shapes of the markers 22.

For the markers 22, a cross shape like one in FIG. 4A or 4B, or a shape having a kinked-line structure (having a point) like one illustrated in FIG. 4C is employed. Meanwhile, a shape having two or more similar structures parallel to each other, like one illustrated in FIG. 4D or 4E, is not used for the markers 22.

Each marker 22 is formed of a substance having a large radiation attenuation coefficient, such as lead, in a shape, illustrated in, for example, FIGS. 4A to 4C, with a size of at least 10 mm or larger, and is attached to a marker holding member 26. Each marker holding member 26 is formed of an X-ray transparent material.

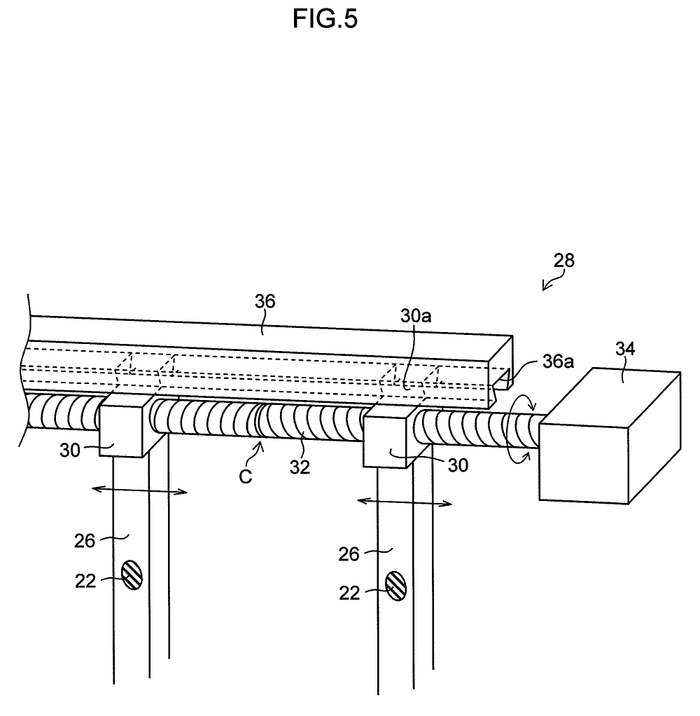

FIG. 5 illustrates a specific example of a marker moving device 28.

As illustrated in FIG. 5, nuts 30 are provided at the upper ends of the marker holding members 26, and a ball screw 32 is inserted into these nuts 30. The ball screw 32 rotates in two directions, i.e., normal and reverse directions, by means of a motor 34. Also, threads running in opposite directions are provided on the right and left sides of the ball screw 32 with the center C as the boundary, and the two marker holding members 26 respectively fit the threads running in the opposites directions.

Accordingly, upon rotation of the ball screw 32, the two marker holding members 26 move in directions opposite to each other, and thus, move close to each other or move away from each other.

Also, a linear movement guide 36 is provided on the upper sides of the nuts provided at the ends of the marker holding members 26, and protrusions 30a provided at the upper portions of the nuts 30 slidably fit in a groove 36a of the linear movement guide 36.

A structure similar to the above is provided at the other (lower) ends of the marker holding members 26, and the two marker holding members 26 can move laterally in parallel to each other by driving the motors 34 of the upper and lower marker moving devices 28 in synchronization with each other by means of the control unit 24.

Also, in order to keep them in parallel to each other during moving, one or two linear movement guides formed of a material having radiotransparency may be provided between the upper and lower portions of the marker holding members 26.

Also, in order to ensure smoother movement of the marker holding members 26, a configuration in which a rack is provided instead of the linear movement guide 36 and pinion gears are provided at the upper portions of the nuts 30 to make the pinion gears engage with the rack and move in rotation may be employed. Otherwise, any known driving device enabling accurate synchronized motions of the upper and lower marker moving devices 28 so as to provide smooth parallel movement of the two marker holding members 26 without occurrence of an imbalance in parallel movement can be employed.

Next, an operation of the present embodiment will be described.

As illustrated in FIG. 1, first, when a subject M stands with his/her back in contact with the screen 18, the positions of the X-ray source 12, the collimator 14 and the FPD 16 according to the subject M are set by an image-taking person. Then, the control unit 24 figures out an X-ray field of view width (irradiated area width) W by calculation as follows.

The field of view width W can be calculated using an image taking distance D, which is the distance from the X-ray source 12 to the FPD 16, a distance f from the X-ray source 12 to the collimator 14, and the aperture width d of the collimator 14.

In other words, the following relationship can be established among these values based on the similarity relationship of the triangles.

f:D=d:W

According to this proportional expression, the field of view width W can be calculated by the following expression.

W=D×d/f

When the field of view width W has been calculated by the control unit 24 as described above, the control unit 24 gives an instruction to the marker moving devices 28 to drive the motors 34 of the upper and lower marker moving devices 28 for the marker holding members 26 in synchronization with each other to move the marker holding members 26 in parallel to each other, thereby positioning the markers 22 at the edge portions of the field of view width W.

In this case, the stop positions of the marker holding members 26 may be determined by, e.g., an encoder provided in each motor 34 illustrated in FIG. 5.

Upon determination of the positions of the markers 22, the control unit 24 controls the X-ray source 12 and the FPD 16 to change the irradiated area and the position of the FPD 16 to take plural images.

The plural images taken are sent from the FPD 16 to the control unit 24, and combined into a long length of image in the control unit 24 based on the markers 22 shown in the overlapping parts of the respective images.

As described above, in the present embodiment, when an image-taking person sets, e.g., the positions of the X-ray source, etc., according to a subject M, the area to be irradiated is automatically calculated, and furthermore, the markers are automatically moved to the positions at the edges of the calculated area to be irradiated.

Accordingly, according to the present embodiment, the markers can reliably be included within the irradiated area regardless of the image taking target, the arrangement of the markers, which provide marks for combining plural images, can easily be performed, and thus, the rate of failure of alignment for image combination to obtain a long length of image can be reduced, enabling provision of a proper automatic image combination result.

Also, since an image-taking person does not need to set the positions of the markers, the time for image taking can be reduced, and thus, the work efficiency can be enhanced, enabling saving trouble in long length imaging, which has conventionally been cumbersome.

Next, a second embodiment of the present invention will be described.

In the present embodiment, an irradiation field lamp that emits visible light is provided at a position that is substantially the same as the position of the X-ray source, each marker holding device is provided with a photosensor to automatically determine a field of view width W, and markers are automatically moved.

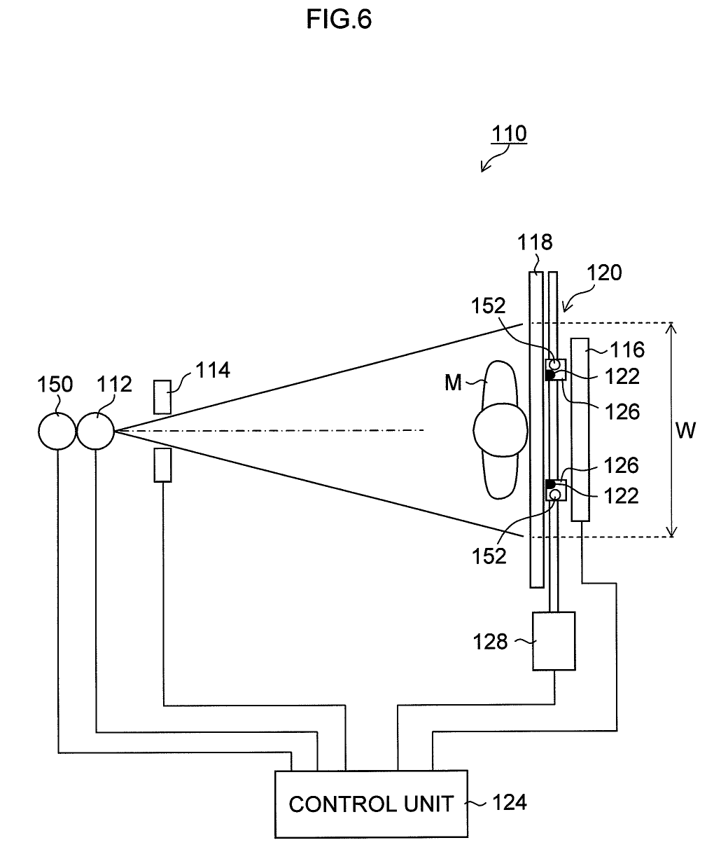

FIG. 6 illustrates a schematic diagram of a configuration of a radiographic apparatus using an image taking support device for radiographic long length imaging according to the present embodiment.

As illustrated in FIG. 6, the image taking support device according to the present embodiment includes an irradiation field lamp 150 provided at a position that is substantially the same as the position of an X-ray source 112, the irradiation field lamp 150 emitting visible light; photosensors 152 provided on marker holding members 126 separately from markers 122, the photosensors 152 being arranged at regular intervals, as with the markers 122, between the markers 122, as components in addition to the components of the first embodiment.

The irradiation field lamp 150 emits visible light from the position that is substantially the same as the position of the X-ray source 112 toward a subject M. The light emitted by the irradiation field lamp 150 is focused by a collimator 114 into a flux of light, which is applied to a screen 118 and a marker device 120, and the area irradiated with the flux of light is the same as an X-ray's field of view.

The photosensors 152 provided on the marker holding members 126 detect a field of view width W by detecting the area irradiated with the irradiation field lamp 150.

Each photosensor 152 is constructed so that current flows in the photosensor 152 upon receipt of light, and upon detection of the current by a control unit 124, the control unit 124 gives an instruction to drive marker moving devices 128. As described above, detection of a field of view width W and movement of marker positions are automatically performed.

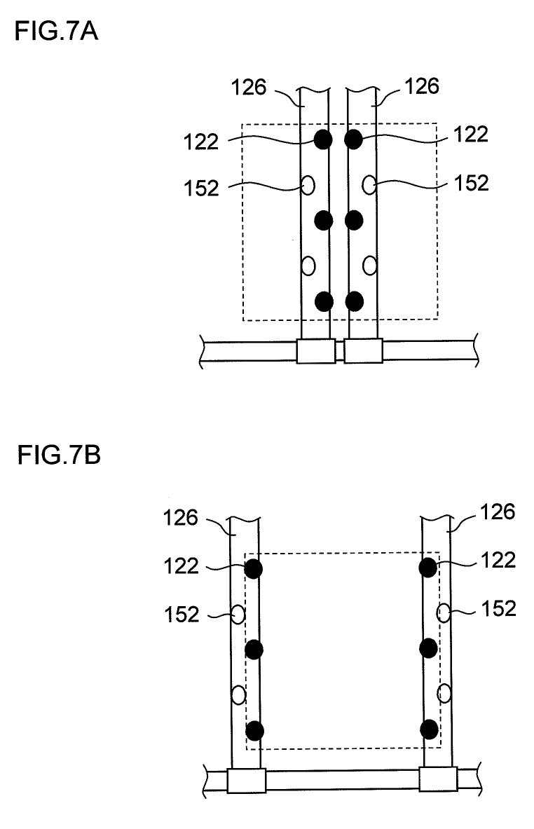

FIGS. 7A and 7B illustrate movement of the marker positions.

First, as illustrated in FIG. 7A, in an initial state, two marker holding members 126 are positioned at the center of the field of view width W indicated with dotted lines.

When light is emitted from the irradiation field lamp 150 and the photosensors 152 provided on the marker holding members 126 detects the light, current flows, and when the current is detected by the control unit 124, the marker moving devices 128 are driven by an instruction from the control unit 124, and as illustrated in FIG. 7B, the two marker holding members 126 laterally move away from each other in parallel to each other.

Then, when the marker holding members 126 come close to the edge portions of the irradiated area indicated with dotted lines, the amounts of light received by the photosensors 152 are reduced, and when the photosensors 152 comes out of the irradiated area, the marker holding members 126 stop there. In this case, it is preferable that: the photosensors 152 are provided on the outer sides of the marker holding members 126; and the markers 122 are provided on the inner sides of the marker holding members.

Consequently, when the photosensors 152 have barely come out of the outer limit of the irradiated area, the markers 122 are positioned just at the edge portions of the irradiated area.

As a result of automatically determining the area to be irradiated and moving the markers as described above, a long-length of image can be taken more smoothly.

The components other than those described above are similar to those in the above-described first embodiment, and thus, they are provided with reference numerals whose last two digits are the same as the reference numerals of the corresponding components in the first embodiment and a detailed description of those components is omitted.

As described above, also in the present embodiment, all the procedures from determination of the area to be irradiated to movement of the markers can be performed automatically, enabling further enhancement of work efficiency in long length imaging.

In the above-described examples, a description has been given for taking an image of a subject in a standing position. When taking an image of a subject in a supine position, an image taking bed is used as a supporting member for a subject instead of a screen, and a configuration including this corresponds to an image taking support device.

Although a detailed description has been given for an image taking support device for radiographic long length imaging according the present invention, it should be understood that the present invention is not limited to the above-described examples and that various modifications and variations may be made within the scope not deviating from the spirit of the present invention.

What is claimed is:

1. An image taking support device for radiographic long length imaging, the image taking support device providing an image of a subject, the image being longer than a length of a maximum field of view of a radiation detector, by combining a plurality of radiographic images each partially overlapping another at the overlapping parts, the device comprising:
a supporting member including a radiotransparent material and supporting the subject;
markers including a substance with a large radiation attenuation coefficient, the markers being movably arranged along a surface of the supporting member supporting the subject so that the markers provide marks for combining the images;
an irradiated area determination device that determines an area to be irradiated with a radiation;
a marker position determining device that determines positions for arranging the markers, based on the determined area to be irradiated; and
a marker moving device that moves the markers to the determined positions of the markers.

2. The image taking support device for radiographic long length imaging according to claim 1, wherein the subject is arranged in a standing position.

3. The image taking support device for radiographic long length imaging according to claim 2, wherein: the markers are arranged substantially parallel to a body axis of the subject; a predetermined number of the markers is arranged on each of two rod-like members that are moved in parallel to each other to widen/narrow the space between the two rod-like members, with a predetermined space between each marker; and the marker moving device is a device that moves the two rod-like members on which the markers are arranged, in parallel to each other.

4. The image taking support device for radiographic long length imaging according to claim 1, wherein the irradiated area determining device determines the area to be irradiated by calculating the area to be irradiated from a distance between a source of the radiation and the radiation detector and the size of an aperture for the radiation.

5. The image taking support device for radiographic long length imaging according to claim 2, wherein the irradiated area determining device determines the area to be irradiated by calculating the area to be irradiated from a distance between a source of the radiation and the radiation detector and the size of an aperture for the radiation.

6. The image taking support device for radiographic long length imaging according to claim 3, wherein the irradiated area determining device determines the area to be irradiated by calculating the area to be irradiated from a distance between a source of the radiation and the radiation detector and the size of an aperture for the radiation.

7. The image taking support device for radiographic long length imaging according to claim 3, further comprising: an irradiation field lamp arranged at a position that is substantially the same as the position of a source of the radiation, the irradiation field lamp applying visible light to the subject; and a plurality of photodetectors arranged on the rod-like members, wherein the irradiated area determining device detects light from the radiation field lamp while moving the rod-like members in parallel to each other, to determine the area to be irradiated.
